(19) United States
(12) Patent Application Publication
(10) Pub. No.: US 2026/0295037 A1
(43) Pub. Date: Oct. 1, 2026

(54) SAPONIN DMLT ADJUVANTS AND RELATED USES

(71) Applicants: Q-VANT BIOSCIENCES, INC., Forestdale, MA (US); THE ADMINISTRATORS OF THE TULANE EDUCATIONAL FUND, New Orleans, LA (US)

(72) Inventors: Elizabeth NORTON, New Orleans, LA (US); Doug KLAIBER, Forestdale, MA (US)

(21) Appl. No.: 19/120,270

(22) PCT Filed: Oct. 12, 2023
(86) PCT No.: PCT/US2023/076673

§ 371 Date: Apr. 11, 2025

Related U.S. Application Data
(60) Provisional application No. 63/415,823, filed on Oct. 13, 2022.

(51) Int. Cl. A61K 39/39; A61K 39/00; A61K 39/08; A61P 31/04

(52) U.S. Cl. CPC A61K 39/39; A61K 39/08; A61P 31/04; A61K 2039/542; A61K 2039/55544; A61K 2039/55577; A61K 2039/572; A61K 2039/575

(57) ABSTRACT

A composition, preferably an immune adjuvant system, for vaccines containing a saponin component and a dmLT component. The combination of the saponin component and the dmLT component shows a synergistic effect in the treatment of various conditions, illnesses and diseases. The methods of use for prophylactic use or therapeutic treatment are disclosed. Exemplary adjuvant compositions include a double-mutant heat-labile toxin adjuvant derived from an Escherichia coli enterotoxin and a saponin, optionally with an additional vaccine component (e.g., an antigen), particularly when used in a vaccine.

### CROSS REFERENCE TO RELATED APPLICATIONS

This application claims the benefit of U.S. Provisional Patent Application No. 63/415,823, filed Oct. 13, 2022, the disclosure of which is incorporated herein by reference.

### FIELD OF THE INVENTION

The present disclosure relates to an immune adjuvant system for vaccines containing a saponin component and a dmLT component, their methods of use for prophylactic use or therapeutic treatment. Exemplary adjuvant compositions include a double-mutant heat-labile toxin adjuvant derived from an Escherichia coli enterotoxin and a saponin, optionally with an additional vaccine component (e.g., an antigen), particularly when used in a vaccine.

### BACKGROUND OF THE INVENTION

The adjuvant dmLT, or more technically LT(R192G/L211A), is an 84-kDa polymeric protein with an AB5 structure composed of an enzymatically active A subunit (28 kDa) noncovalently associated with a pentameric B subunit (consisting of five 11.5-kDa monomers). dmLT is distinguished from its parent molecule heat-labile enterotoxin (LT) by the substitution of two residues in the A subunit, a glycine for an arginine at amino acid 192 (R192G) and an alanine for a leucine at amino acid 211 (L211A). The ribbon diagram of dmLT can be extrapolated from the crystal structure of the partially cleaved LT toxin (ref 1, 2), although there may be as-yet-unresolved changes in three-dimensional (3D) structure due to the amino acid substitutions in the A subunit. dmLT is an adjuvant that enhances vaccine-specific systemic and mucosal immune responses following mucosal or parenteral delivery. Studies indicate four main features that define dmLT compared with other adjuvant systems.
(1) dmLT promotes immunity to antigens that are codelivered after simply admixing dmLT and the antigen in aqueous buffer. Thus, unlike many depot-type adjuvants, such as aluminum hydroxide, no advanced preparation or absorption is required to formulate the antigen/adjuvant vaccine. dmLT can be formulated with the antigen at either the point of manufacture or the point of delivery.
(2) Through the combined action of dmLT's immunostimulatory properties and universal cell binding, uptake of codelivered antigens is enhanced and mucosal immunity is promoted. This enables the delivery of immunization formulations (most strikingly for subunit vaccines) at previously inaccessible sites, such as in oral (p.o.), sublingual (s.l.), transcutaneous (t.c.i.), etc., delivery. Many of these approaches are needle free and have the potential to increase ease of administration and compliance and lower the risk of disease outbreaks from unsafe injections (3-6).
(3) Unlike other adjuvants, such as aluminum hydroxide or many Toll-like receptor (TLR)-based adjuvants (e.g., monophosphoryl lipid A [MPL] and CpG), dmLT induces strong interleukin-17 (IL-17) recall cytokine secretion and antigen-specific Th17 responses after parenteral or mucosal immunization (7-15). This is a newly appreciated arm of the adaptive immune response that is critical in protection from pathogens, particularly in preventing infections in mucosal tissue and control of bacterial infections (16). In addition, IL-17 secretion enhances the availability of mucosal antibodies by upregulating polymeric Ig receptor levels in epithelial cells, increasing transport of secretory IgA (sIgA) into the lumen of mucosal tissue, and promoting T-independent B-cell differentiation into IgA-secreting cells (17-20).
(4) Last, dmLT promotes the development of mucosal immune responses following parenteral immunization.

The adjuvant effect of saponins was first reported in 1925, when it was shown that the addition of bread crumbs, tapioca, saponin and “starch oil” to antigenic preparations greatly enhanced antibody responses to diphtheria or tetanus. In 1951, Espinet used a crude commercially available saponin preparation to increase the potency of foot-and-mouth disease vaccines. Further in 1974, Dalsgaard successfully isolated saponin Quil A from the bark of the South-American Tree Quillaja saponaria Molina and found that Quil A stimulated both humoral and cellular immunity, as well as induced differential antibody isotypes. Since then, the Quil AR has been commercialized and has gained widespread use in veterinary vaccines and pre-clinical studies. Additional studies showed its effects when co-formulated with aluminum salts, liposomes and oil-in-water emulsions, and with amphipathic proteins and lipids forming detergent/lipid/saponin complexes termed immune-stimulating complexes (ISCOMs).

Quil A is a heterogeneous product, consisting of up to 23 different saponin peaks detectable by HPLC and its toxicity when injected parentally precluded its use in human vaccines. Due to the fact that Quil A is a mixture, a further study was performed by Kensil et al. in 1991, in which 10 of RP-HPLC fractions from aqueous extract of Q. saponaria bark treated by ultrafiltration were tested and found the fractions QS-7, QS-17, QS-18 and QS-21 to be particularly potent. However, their toxicity when administered parentally (injected) varies considerably. QS-18, the major component of Q saponaria, was found to be highly toxic in mice, while QS-7 and QS-21 shows far less toxicity.

Saponin fractions such as QS-21, are currently being utilized in the GSK Shingrix® shingle vaccine and the Mosquirix® malaria vaccine and saponin Fractions A and C are currently being used in the Matrix-M® adjuvant in the Novavax Covid-19 vaccine in humans. Saponin-based adjuvants are also being investigated in numerous other human indications (i.e., TB, RSV, cancer, etc.).

Purified saponins have proven to be safe and effective when injected and have significantly enhanced the efficacy of some oral vaccines under clinical investigation. The toxicity of the food-grade saponin (FGS) extract from Quillaja saponaria has limited its use as a parenteral adjuvant; however, this toxicity is abated when delivered orally.

FGS extract is commonly used within the global food and beverage industries and is Generally Recognized As Safe (GRAS) by FDA and in Europe by EFSA's Panel on Food Additives and Nutrient Sources (ANS). FGS has no documented toxicity in humans at the present levels of consumption in both adults and in infants of 1.5 mg/kg/day or approximately 100 mg of pure saponins in adults and 12 mg in infants. To put this into perspective, calculating the amount of QS-21 safely ingested as FGS in 100 mg of pure saponins equates to approximately 2.8% or 2.8 mg of QS-21 which is approximately 60 times the amount of QS-21 found in the Shingrix® vaccine (50 mcg) and 120 times more than in the Mosquirix® vaccine (25 mcg).

Food-grade saponins (FGS) extracts are defined as either Type 1 and 2 extracts and have significant potential advantages for use with vaccines and provide a broad adjuvant effect due to the multiple saponin components.

Despite the previous literature wherein Saponins have a lengthy adjuvant history, and knowing that several saponin fractions have been and currently are being utilized in commercial vaccines in humans, the need still exists for a vaccine adjuvant system as set forth by the present disclosure, which provides a vaccine adjuvant system derived from Quillaja saponaria and dmLT for use in vaccines. It is particularly noted that all current vaccines using saponins are injected vaccines, and no mucosal saponin adjuvant currently exists. In the present invention, the use of both the mucosal saponin adjuvants and dmLT adjuvants show a synergistic effect that was unexpected and that has not been described in the state of the art.

### SUMMARY OF THE INVENTION

The present disclosure provides compositions and methods of using compositions for the treatment of a disease or disorder, or illness or condition. As described herein, the composition or compositions (e.g., a “system” as used herein) may include one or more or all of a saponin, and a heat-labile toxin including, for example, dmLT, and their use and methods for prophylactic use or therapeutic treatment. Exemplary adjuvant compositions include a double-mutant heat-labile toxin adjuvant derived from an Escherichia coli enterotoxin and a saponin, optionally with a vaccine or vaccine component (e.g., an antigen). In one or more embodiments, the composition may include (i) a polymeric protein containing at least a portion of dmLT, and (ii) a saponin compound derived from Quillaja saponaria. By the phrase “at least a portion,” it is meant that the polymeric protein is a detoxified enterotoxin derived from Escherichia coli with at least 80% identity to the amino acid sequence of dmLT, wherein the detoxified enterotoxin is an adjuvant that retains the immunological activities of dmLT.

In one embodiment, the present disclosure provides a vaccine adjuvant system comprising (i) a composition comprising an dmLT, (ii) a composition comprising a saponin, and optionally (iii) a vaccine component. In other embodiments, the vaccine adjuvant system may include the composition above which includes an effective dose of the saponin compound derived from Quillaja saponaria and an effective dose of at least a portion of dmLT.

In another embodiment, the present disclosure provides a vaccine containing the synergistic vaccine adjuvant system as above, wherein said system comprises an effective dose of a Quillaja saponaria composition and an effective dose of a dmLT. An alternative embodiment of a vaccine would further include the vaccine adjuvant system above that comprises or encodes at least one antigen. The vaccine may further comprise a polypeptide, a nucleic acid, a polysaccharide, a polysaccharide-polypeptide conjugate, a live-attenuated or inactivated bacterium, a toxoid, a live-attenuated or inactivated virus, a virus-like particle, a viral vector, and/or combinations thereof. Some embodiments of the vaccine may also comprise or encode a bacterial, viral or fungal antigen.

In another embodiment, a kit comprising any of the vaccine adjuvant systems or vaccines above may be provided.

In still another embodiment, a method of generating an immune response in a subject comprising administering to the subject the vaccine adjuvant system or a vaccine as above is disclosed. In at least one embodiment, the step of administering is by an oral route or a sublingual route. In at least one embodiment of the method, the immune response is a B cell response. In one or more other embodiments, the immune response is the generation of CD4+ T cells. In still one or more other embodiments, the immune response is the generation of CD8+ T cells.

In still another embodiment, the present disclosure provides a method of treating of an illness, disease, or a condition, comprising administering a vaccine containing a vaccine adjuvant system comprising an effective dose of a saponin compound derived from Quillaja saponaria; an effective dose of at least a portion of dmLT; and, optionally, an effective dose of one or more vaccine components associated with the illness, disease, or condition.

In further embodiments, the present invention provides for use of the composition of in a vaccine. In other embodiments, the present invention provides for the use the vaccine adjuvant system in a vaccine. And in other embodiments, the present invention provides use of the vaccine to treat an illness, disease, or condition.

### BRIEF DESCRIPTION OF THE DRAWING

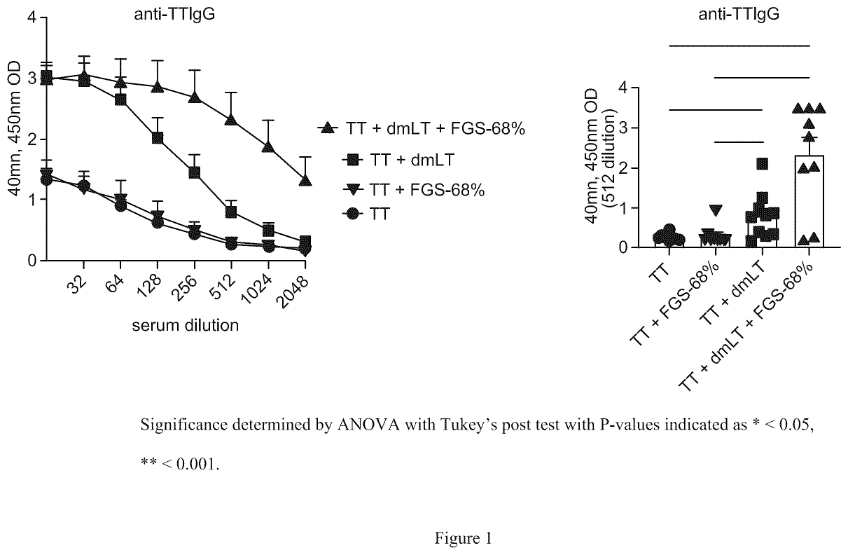

The drawing figure (FIG. 1) shows an oral immunization with combined dmLT and saponin adjuvant improves immunity to tetanus toxoid (TT) antigen. Analysis of anti-TT IgG by ELISA with raw optical density values shown left, and compiled shown right. Significance is determined by ANOVA with Tukey's post-test with P-values indicated as *<0.05, **<0.001.

### DETAILED DESCRIPTION

The present disclosure provides a composition, such as, for example, a synergistic vaccine adjuvant system, comprising a saponin compound derived from Quillaja saponaria and dmLT for use in vaccines and the like.

In one of the embodiments of the present disclosure, a synergistic vaccine adjuvant system for use in vaccines is described, wherein said vaccine adjuvant system comprises an effective dose of a Quillaja saponaria compound and an effective dose of a dmLT for use as an immune adjuvant system for vaccines. In one or more other embodiments of the present disclosure, the use of a vaccine adjuvant system is described, wherein the Quillaja saponaria compound and dmLT within the adjuvant system are used as an immune adjuvant system for vaccines. In still one or more other embodiments of the present disclosure, a therapeutic treatment method is described, wherein said method comprises administering a vaccine which includes an effective dose of a Quillaja saponaria compound, an effective dose of dmLT, and an active ingredient or vaccine component, such as a sterol, a phospholipid, and/or an antigen, for the prevention or treatment of an illness, disease, or a condition. The adjuvant system for vaccines described herein can be administered in any of a number of ways, including but not limited to the following routes: oral, sublingual, subcutaneous, parenterally, rectally, otic pathway, nasal route, cutaneous route, transdermal route, or combinations thereof.

Saponin compounds are raw materials and may be obtained from the bark or, in some cases, the whole biomass of the tree Quillaja saponaria Molina. The term “saponin” as used herein includes glycosidic triterpenoid compounds (also known as triterpene glycoside compounds) which produce foam in aqueous solution, have hemolytic activity in most cases, and possess immune adjuvant activity. The term “saponin” also encompasses biologically active fragments of the above compounds. It will be appreciated that the term “QS” refers to Quillaja saponin.

Quillaja saponins are structurally distinct from the saponins derived from other plant species. Two structural features that distinguish Quillaja saponaria saponins from those of other plant species are a fatty acid domain and a triterpene aldehyde at carbon 4 of the triterpene.

In the present disclosure, the Quillaja saponaria compounds can be selected form, but not necessarily limited to: Crude saponin extracts, Type 1 Extract, Type 2 Extract, QS-7, QS-8, QS-17, QS-18, QS-21, QS-21 Fraction A, QS-21 Fraction C, or combinations thereof. The four most predominant identified and purified Quillaja saponins are QS-7, QS-17, QS-18, and QS-21. As noted above, these adjuvant saponins have been identified and purified from an aqueous extract of the bark of the South American tree, Quillaja saponaria Molina, and have generally been purified by HPLC and low-pressure silica chromatography and were found to be adjuvant active, although differing in biological activities such as hemolysis and toxicity. More recently, the obtention of these saponins from the entire biomass of the Quillaja saponaria Molina tree has provided for significant increases in the available raw material, triggering an increase in production capacities, reducing costs and making the product sustainable, since the extraction of biomass does not cause the death of the tree or stress over the native Chilean forest.

Beyond the saponin compounds of the present invention, the other component of the adjuvant compositions provided herein is a detoxified enterotoxin adjuvant derived from Escherichia coli referred to in the literature as dmLT. The detoxified enterotoxin adjuvant exemplified herein was originally described in U.S. Pat. No. 6,033,673. Referred to as “LT(R192G/L211A)” in that patent and referred to as “dmLT” herein, the detoxified enterotoxin adjuvant is a genetically distinct mutant of the E. coli heat-labile enterotoxin (LT) which through modification of the arginine at position 192 to glycine and the modification of the leucine at position 211 to arginine, has lost the trypsin sensitive site joining the A1 and A2 subunits, rendering the molecule non-toxic but still able to act as an immunological adjuvant.

dmLT can be produced by methods standard in the art. For example, plasmid pECD403, described in Example 6.1 of U.S. Pat. No. 6,033,673, can be utilized to produce substantially pure LT(R 192G/L211A) (dmLT) in E. coli. dmLT can be isolated by agarose affinity chromatography from bacteria expressing an dmLT-encoding plasmid. Alternate methods of purification standard in the art can be used to purify dmLT.

Other detoxified enterotoxins with equivalent immunological activities can be made and used in compositions and methods disclosed herein by those skilled in the art. For example, detoxified enterotoxins with at least 80%, 90%, 91%, 92%, 93%, 94%, 95%, 96%, 97%, 98% or 99% identity to the amino acid sequence of the dmLT adjuvant that retain the immunological activities of dmLT are contemplated.

Compositions, and preferably adjuvant compositions, provided herein comprise a detoxified bacterial endotoxin adjuvant and saponin compound adjuvant. The adjuvant compositions can comprise a dmLT adjuvant and/or a Quillaja saponaria compound including, but not limited to, Crude saponin extracts, Type 1 Extract, Type 2 Extract, QS-7, QS-8, QS-17, QS-18, QS-21, QS-21 Fraction A, QS-21 Fraction C, or combinations thereof.

The adjuvant compositions, including the adjuvant systems described herein, may be administered as separate compositions with or without a vaccine composition. In other embodiments, the adjuvant compositions may be administered in the same/one composition, or optionally, separate from an optional one or more vaccine component.

The adjuvants provided herein are present in a composition in an amount between the ranges of 0.1-1000 μg/dose for each individual adjuvant (dmLT and saponin). Doses may be adjusted depending upon the body mass, body area, weight, blood volume of the subject, or route of delivery. It will be evident to those skilled in the art that the number and frequency of administration will be dependent upon the response of the host. As described herein, the appropriate dose may also depend upon the subject's condition, that is, stage of the disease, general health status, as well as age, gender, and weight, and other factors familiar to a person skilled in the medical art. Thus, by the term “effective dose,” it is meant a dosage believed by one of ordinary skill having knowledge of the individual subject or host as being suitable and sufficient to have a positive effect on the individual in the treatment of the condition, illness or disease.

The adjuvant compositions may be in any form which allows for the composition to be administered to a subject. For example, the adjuvant composition may be in the form of a solid, liquid or gas (aerosol). The pharmaceutical compositions may be administered by any route. Typical routes of administration include, without limitation, oral, sublingual, buccal, topical, parenteral (including intradermal, subcutaneous, percutaneous, intravenous, intramuscular, intrasternal, intracavernous, intrameatal, intratumoral, intracranial, intraspinal or intraurethral injection or infusion), rectal, vaginal, intranasal (e.g., as a spray) and intrapulmonary administration. The term “parenteral” as used herein includes, but is not limited to, iontophoretic, sonophoretic, thermal, passive transdermal, and microneedle administration and also intradermal/subcutaneous injections, intravenous, intramuscular, intrasternal, intracavernous, intrathecal, intranodal, intrameatal, intraurethral, intratumoral injection or infusion techniques. An adjuvant composition as provided herein can be administered intradermally by a technique selected from iontophoresis, microcavitation, sonophoresis or microneedles.

The adjuvant compositions may further comprise at least one physiologically (or pharmaceutically) acceptable or suitable excipient. Any physiologically or pharmaceutically suitable excipient or carrier (i.e., a non-toxic material that does not interfere with the activity of the active ingredient) known to those of ordinary skill in the art for use in pharmaceutical compositions may be employed in the compositions provided herein. Exemplary excipients include diluents and carriers that maintain stability and integrity of proteins. Excipients for therapeutic use are well known.

“Pharmaceutically acceptable carriers” are also well known in the pharmaceutical art. For example, sterile saline and phosphate buffered saline at physiological pH may be used. Preservatives, stabilizers, dyes and even flavoring agents may be provided in the pharmaceutical composition. For example, sodium benzoate, sorbic acid and esters of p hydroxybenzoic acid may be added as preservatives. In addition, antioxidants and suspending agents may be used.

Similarly, “Pharmaceutically acceptable salts” refers to salts of a compounds derived from the combination of such compounds and an organic or inorganic acid (acid addition salts) or an organic or inorganic base (base addition salts). The adjuvant compositions provided herein may be used in either the free base or salt forms.

The adjuvant composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more adjuvants in aerosol form may hold a plurality of dosage units.

A liquid adjuvant composition can be provided herein, whether in the form of a solution, suspension or other like form, and may include one or more of the following carriers or excipients: sterile diluents such as water for injection, saline solution, preferably physiological saline, Ringer's solution, isotonic sodium chloride, fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

An adjuvant composition provided herein can comprise a stable aqueous suspension of less than 0.2 μM and further comprises at least one component selected from the group consisting of phospholipids, fatty acids, surfactants, detergents, saponins, fluorodated lipids, and the like. Such a stable aqueous formulation may be a micellar formulation.

An adjuvant composition provided herein can be formulated in a manner which can be aerosolized, either as a powder or liquid formulation.

It may also be desirable to include other components in the adjuvant composition, such as including but not limited to water-in-oil emulsions, biodegradable oil vehicles, oil-in-water emulsions, liposomes, micellar components, microparticles, biodegradable microcapsules, and liposomes.

Adjuvant compositions provided herein can comprise a stable oil-in-water emulsion and a metabolizable oil. The meaning of the term metabolizable oil is well known in the art. Metabolizable can be defined as “being capable of being transformed by metabolism”. The oil may be any plant oil, vegetable oil, fish oil, animal oil or synthetic oil, which is not toxic to the recipient and is capable of being transformed by metabolism. Nuts (such as peanut oil), seeds, and grains are common sources of vegetable oils. Synthetic oils may also be used.

Additional immunostimulatory substances may be included in the adjuvant compositions provided herein and may include N-acetylmuramyl-L-alanine-D-isoglutamine (MDP), glucan, IL-12, GM CSF, interferon-y and IL-12.

While any suitable carrier known to those of ordinary skill in the art may be employed in the adjuvant compositions provided herein, the type of carrier will vary depending on the mode of administration and whether a sustained release is desired. Biodegradable microspheres (e.g., polylactic galactide) may also be employed as carriers for the adjuvant compositions provided herein. Suitable biodegradable microspheres are known in the art. In this regard, it is preferable that the microsphere be larger than approximately 25 microns.

Adjuvant compositions provided herein may also contain diluents such as buffers, antioxidants such as ascorbic acid, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Adjuvants may be formulated as a lyophilizate using appropriate excipient solutions (e.g., sucrose) as diluents.

In one or more embodiments of the present invention, the adjuvant compositions may further be contained in a vaccine. Where a vaccine is provided, the vaccine may further comprise or encode an antigen.

The adjuvant compositions and methods provided herein are intended for use in a subject, including humans and other animals. Vaccines contemplated for use with adjuvant compositions may comprise or encode an antigen. The vaccines containing the adjuvant composition therein may also comprise or encode, for example, a bacterial, viral or fungal antigen.

Antigens contemplated by the disclosure, as examples and not by way of limitation, include antigens from pathogenic strains of bacteria (including, but not limited to, Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Bordetella pertussis, Vibrio cholerae, Escherichia coli, Pseudomonas aeruginosa, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Salmonella typhi, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., Helicobacter pylori); pathogenic fungi (Coccidioides immitis, Aspergillus fumigatus, Candida albicans, Blastomyces dermatitidis, Cryptococcus neoformans, Histoplasma capsulatum); protozoa (Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria); or Helminths (Enterobius vermicularis, Trichuris trichiura, Ascaris lumbricoides, Trichinella spiralis, Strongyloides stercoralis, Schistosoma japonicum, Schistosoma mansoni, Schistosoma haematobium, and hookworms) either presented to the immune system in whole organism form or in part isolated from media cultures designed to grow said organisms which are well known in the art, or protective antigens from said organisms obtained by genetic engineering techniques or by chemical synthesis.

Other antigens contemplated include, for example, antigens from pathogenic viruses (e.g., Poxviridae, Herpesviridae, Herpes Simplex virus 1, Herpes Simplex virus 2, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Hepatitis A virus, Hepatitis B virus, Hepatitis C virus, Hepatitis E virus, Non-A/Non-B Hepatitis virus, Rhinoviridae, Coronaviridae, Rotoviridae, and Human Immunodeficiency Virus) (e.g., rabies virus, herpesviruses, such as herpes simplex virus (HSV) type 2, HSV type 1, human cytomegalovirus, Epstein-Barr virus, and varicella zoster virus (VZV), human papillomavirus (HPV), Human T-cell lymphotropic virus type 1, rotavirus, norovirus, hepatitis A virus, hepatitis B virus, hepatitis C virus, influenza virus, polio virus, Japanese encephalitis virus, measles virus, mumps virus, rubella virus, yellow fever virus, varicella virus, dengue virus, rotavirus, paniovirus, human immunodeficiency virus-1, ebolaviruses, such as Ebola Sudan virus, Marburg virus, hantavirus, norovirus, Zika virus, West Nile virus, hantavirus, Lassa virus, Lymphocytic choriomeningitis virus, Nipah virus, Rift Valley fever virus, Middle East Respiratory Syndrome Coronavirus, SARS coronavirus, SARS coronavirus 2, Crimean-Congo hemorrhagic fever virus, enteroviruses, and noroviruses) either presented to the immune system in whole or in part isolated from media cultures designed to grow such viruses which are well known in the art, or antigens therefrom obtained by genetic engineering techniques or by chemical synthesis.

Examples of vaccines contemplated include, but are not limited to, influenza vaccine, pertussis vaccine, diphtheria and tetanus toxoid combined with pertussis vaccine, hepatitis A vaccine, hepatitis B vaccine, hepatitis C vaccine, hepatitis E vaccine, Japanese encephalitis vaccine, herpes vaccine, measles vaccine, rubella vaccine, mumps vaccine, mixed vaccine of measles, mumps and rubella, papillomavirus vaccine, parvovirus vaccine, respiratory syncytial virus vaccine, Lyme disease vaccine, polio vaccine, varicella vaccine, gonorrhea vaccine, schistosomiasis vaccine, rotavirus vaccine, Mycoplasma vaccine pneumococcal vaccine, meningococcal vaccine, Campylobacter vaccine, Helicobacter vaccine, cholera vaccine, enterotoxigenic E. coli vaccine, enterohemmorgagic E. coli vaccine, Shigella vaccine, Salmonella vaccine and others. These are produced by known common processes. In general, such vaccines comprise either the entire organism or virus grown and isolated by techniques well known to the skilled artisan, or comprise relevant antigens of these organisms or viruses which are produced by genetic engineering techniques or chemical synthesis.

It will be appreciated that still other compositions as provided herein may be in a kit. In the kit, the components of the adjuvant composition can already be mixed together for administration, or the components can be separate in the kit and administered separately as directed.

Finally, LT(R 192G/L211A), or dmLT, is the product of more than 35 years of research on the use of bacterial ADP-ribosylating enterotoxins as adjuvants. dmLT has recently had success in Phase 1 and 2 clinical trials for ETEC and polio virus, though success for oral delivery seems to be dependent upon the immunogenicity of the antigen. Both adjuvants have been pursued independently for various vaccines and delivery routes but have not been pursued together in any context. In preliminary studies, it has been observed that synergistic adjuvant activity unexpected ex when dmLT and saponins are co-administered by oral and sublingual vaccination. Among the projects being evaluated is one to evaluate the combination of saponin and dmLT to provide a saponin dmLT adjuvant (SDA) for oral or sublingual delivery. Testing is currently underway for optimizing SDA in globally relevant vaccines, including an adjuvanted subunit ETEC and Campylobacter vaccine (ASEC) targeting bacterial enteric infections and inactivated polio vaccine. Projects are also exploring formation preparations specific to gastrointestinal delivery and conduct IND-enabling studies such as GLP and cGMP manufacturing, toxicology, and stability testing. It is believed that the present invention will help to define the SDA adjuvant platform and formulation that provides potent systemic immunity and/or sustained mucosal immunity.

The following example is exemplary in nature and the present invention is not limited thereto. Rather, as is noted above, the present invention relates to compositions and methods of using the compositions for the treatment of a disease or disorder, or illness or condition. The composition or compositions (e.g., a “system” as used herein) may include one or more of a Quillaia saponin, and a heat-labile toxin including, for example, dmLT, and their use and methods for prophylactic use or therapeutic treatment.

### EXAMPLE 1

An oral administration of the combination of saponin and dmLT in with Tentanus Toxoid was prepared, wherein balb/c mice were then immunized on days 0, 7, 14 in the following groups:
1) 100 ug TT alone
2) 100 ug TT with 25 ug dmLT
3) 100 ug TT with 500 ug food grade saponin Type 2 extract and
4) 100 ug TT with a combination of 25 ug dmLT and 500 ug food grade saponin Type 2 extract
   Serum was collected on day 21 for analysis of anti-TT IgG by ELISA.

Results as shown in FIG. 1 show a synergistic and unexpected effect when the two adjuvants are combined versus no adjuvant and when compared to the individual adjuvants.

1. A composition comprising (i) a polymeric protein containing at least a portion of dmLT, and (ii) a saponin compound derived from Quillaja saponaria.

2. The composition of claim 1, wherein, the polymeric protein is a detoxified enterotoxin derived from Escherichia coli with at least 80% identity to the amino acid sequence of dmLT, wherein the detoxified enterotoxin is an adjuvant that retains the immunological activities of dmLT.

3. The composition of claim 1, wherein the saponin compound is selected from the group consisting of crude saponin extracts, Type 1 Extract, Type 2 Extract, QS-7, QS-8, QS-17, QS-18, QS-21, QS-21 Fraction A, QS-21 Fraction C, and combinations thereof.

4. A vaccine adjuvant system comprising the composition of claim 1, further comprising an effective dose of the saponin compound derived from Quillaja saponaria and an effective dose of at least a portion of dmLT.

5. A vaccine comprising the vaccine adjuvant system of claim 4 that comprises or encodes at least one antigen.

6. The vaccine of claim 5, further comprises a polypeptide, a nucleic acid, a polysaccharide, a polysaccharide-polypeptide conjugate, a live-attenuated or inactivated bacterium, a toxoid, a live-attenuated or inactivated virus, a virus-like particle, or a viral vector.

7. The vaccine according to claim 4, wherein the vaccine comprises or encodes a bacterial, viral or fungal antigen.

8. The vaccine according to claim 7, wherein the antigen is selected from the group consisting of antigens from (1) pathogenic strains of bacteria, wherein the strains of bacteria are selected from Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Bordetella pertussis, Vibrio cholerae, Escherichia coli, Pseudomonas aeruginosa, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Salmonella typhi, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori;(2) pathogenic fungi, wherein fungi is selected from the group consisting of Coccidioides immitis, Aspergillus fumigatus, Candida albicans, Blastomyces dermatitidis, Cryptococcus neoformans, Histoplasma capsulatum; (3) protozoa selected from the group consisting of Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria; (4) Helminths selected from the group consisting of Enterobius vermicularis, Trichuris trichiura, Ascaris lumbricoides, Trichinella spiralis, Strongyloides stercoralis, Schistosoma japonicum, Schistosoma mansoni, Schistosoma haematobium, and (5) hookworms.

9. The vaccine according to claim 7, wherein the antigen is selected from antigens from pathogenic viruses, wherein the viruses include any one of Poxviridae, Herpesviridae, Herpes Simplex virus 1, Herpes Simplex virus 2, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Hepatitis A virus, Hepatitis B virus, Hepatitis C virus, Hepatitis E virus, Non-A/Non-B Hepatitis virus, Rhinoviridae, Coronaviridae, Rotoviridae, and Human Immunodeficiency Virus, selected from rabies virus, herpesviruses, such as herpes simplex virus (HSV) type 2, HSV type 1, human cytomegalovirus, Epstein-Barr virus, and varicella zoster virus (VZV), human papillomavirus (HPV), Human T-cell lymphotropic virus type 1, rotavirus, norovirus, hepatitis A virus, hepatitis B virus, hepatitis C virus, influenza virus, polio virus, Japanese encephalitis virus, measles virus, mumps virus, rubella virus, yellow fever virus, varicella virus, dengue virus, rotavirus, paniovirus, human immunodeficiency virus-1, ebolaviruses, such as Ebola Sudan virus, Marburg virus, hantavirus, norovirus, Zika virus, West Nile virus, hantavirus, Lassa virus, Lymphocytic choriomeningitis virus, Nipah virus, Rift Valley fever virus, Middle East Respiratory Syndrome Coronavirus, SARS coronavirus, SARS coronavirus 2, Crimean-Congo hemorrhagic fever virus, enteroviruses, and noroviruses.

10. A kit further comprising the vaccine according to claim 5.

11. A method of generating an immune response in a subject comprising administering to the subject the vaccine adjuvant system of claim 4.

12. The method of claim 11 wherein the step of administering is by an oral route or a sublingual route.

13. The method of claim 11, wherein the immune response is a B cell response.

14. The method of claim 11, wherein the immune response is the generation of CD4+ T cells.

15. The method of claim 11, wherein the immune response is the generation of CD8+ T cells.

16. A method of treating of an illness, disease, or a condition, comprising administering a vaccine containing a vaccine adjuvant system comprising an effective dose of a saponin compound derived from Quillaja saponaria; an effective dose of at least a portion of dmLT; and, optionally, an effective dose of one or more vaccine components associated with the illness, disease, or condition.

17. (canceled)

18. (canceled)

19. (canceled)

20. A method of generating an immune response in a subject comprising administering to the subject the vaccine of claim 5.

21. The method of claim 20, wherein the step of administering is by an oral route or a sublingual route.

22. The method of claim 20, wherein the immune response is a B cell response.
